(19) United States
(12) Patent Application Publication
(10) Pub. No.: US 2013/0331657 A1
(43) Pub. Date: Dec. 12, 2013

(54) SELF-POWERED LIGHTING SYSTEM FOR USE WITH AN ELECTROSURGICAL PENCIL

(71) Applicants: Kenneth Basson, Simi Valley, CA (US); Shawna Basson, Simi Valley, CA (US); Robert J. Netzel, SR., Simi Valley, CA (US); Sheldon Denst, Simi Valley, CA (US)

(72) Inventors: Kenneth Basson, Simi Valley, CA (US); Shawna Basson, Simi Valley, CA (US); Robert J. Netzel, SR., Simi Valley, CA (US); Sheldon Denst, Simi Valley, CA (US)

(21) Appl. No.: 13/490,462

(22) Filed: Jun. 7, 2012

(51) Int. Cl. A61B 1/06

(52) U.S. Cl. 600/249

(57) ABSTRACT

A self-powered lighting system that mounts to an electrosurgical pencil that is used to cauterize human tissue during a medical surgical procedure. The self-powered lighting system of the present invention illuminates the affected region during the medical procedure and is uniquely configured to meet the strict water-proofing and sanitizing requirements of a surgery room setting. The self-powered lighting system of the present invention is powered by a simple rotation of a main body relative to a lens cover to connect a battery to a light source that emanates light. The lens is shaped to properly distribute and direct the light to the affected region.

### FIELD OF THE INVENTION

This invention relates to a self-powered lighting device that illuminates the affected regions during a medical surgical procedure. More particularly, the invention relates to a lighting device that is mounted to an electrosurgical pencil used to cauterize a patient's tissue during a medical surgical procedure.

### DESCRIPTION OF PRIOR ART

Electrosurgical pencils have become widely used by surgeons in recent years. A number of electrosurgical pencils have been developed whereby an active electrode is attached to an insulated handle and a high frequency current is applied thereto. An electrosurgical pencil is an instrument that is used to cauterize, coagulate, and/or cut tissue. Electrosurgical pencils that are found to be particularly safe and effective for surgical applications are those which incorporate finger actuated switches, those which allow for readily interchangeable electrodes, those which are water resistant, and those which are thin and properly balanced for close surgical use where a certain “feel” is necessary for the surgeon to properly use the instrument. The shape of these safe and effective electrosurgical pencils is particularly slender and resembles a writing pencil.

Electrosurgical pencils are used by medical professionals in various types of surgical procedures. A most common surgical procedure is cauterizing a patient's tissue. Typically, cauterizing is employed to sterilize and cut tissue as well as to kill certain tissue that has been infected with disease such as cancer and the like. To accurately perform the surgical procedure, the medical professional needs to be able to illuminate and view the local tissue area to be cauterized. Thus, it is imperative that adequate lighting be provided to the affected regions during the surgical procedure. However, overhead surgery room lighting is rarely sufficient. Accordingly, various types of supplemental lighting equipment have been developed that suits different medical illumination requirements.

The incorporation of a lighting source to a surgical instrument is well known in the art. Most existing surgical light sources involve fiber optic cable or bundles that are permanently attached to a particular surgical instrument with one end of the fiber optic cable or bundle connected to a source of illumination. These types of surgical instrument lights are disclosed in U.S. Pat. No. 6,585,727; U.S. Pat. No. 5,376,087; and U.S. Pat. No. 4,688,569. More recent designs of surgical instrument lights have incorporated an LED light source within the surgical instrument and powered by the electrical current that energizes the surgical instrument itself. These types of surgical instrument lights are disclosed in U.S. Pat. Appl. No. 2007/0049927 and U.S. Pat. Appl. No. 2010/0125172. Furthermore, U.S. Pat. No. 6,428,180 (hereinafter referred to as “the '180 patent”) discloses a lighting unit that is detachably mountable in a variety of ways either to a user-selected location on any suitable surgical instrument or at the user's option to an adjacent location within the confined space to facilitate the lighting and viewing thereat. Although, this '180 patent is the closest prior art to the current invention that the inventor has been able to identify, it fails to provide an inexpensive and low profile unit that is waterproof and can be sanitized effectively for use in a surgery room. The '180 patent also fails to teach a unit that is specifically designed for electrosurgical pencils so as to provide the best mounting location and light distribution for cauterizing a patient's tissue. Instead, the '180 patent broadly describes methods of attaching the lighting unit to any surgical tool at any location. The teachings of the '180 patent are overly broad and ineffective at providing a proper lighting solution specific for use in a sanitized surgery room setting and fitted for an electrosurgical pencil.

Accordingly, there is still a need for a light source used in conjunction with the insulated handle of an electrosurgical pencil that effectively illuminates the affected area and that meets the strict waterproofing and sanitizing requirements of a surgery room setting.

### SUMMARY OF THE INVENTION

Accordingly, the present invention has been made in view of the above-mentioned disadvantages occurring in the prior art. The present invention is a self-powered lighting system that is mounted to an electrosurgical pencil to illuminate the affected region during the surgical procedure of cauterizing a patient's tissue.

It is therefore a primary object of the present invention to provide adequate illumination to the affected region without interfering with the use of the electrosurgical pencil by a medical professional.

Another object of the present invention is to provide a self-powered lighting device that is removably mountable to an electrosurgical pencil without any wiring.

Yet another object of the present invention is to provide a self-powered lighting device that is water-resistant and can be properly sanitized for a surgery room setting.

A still further object of the present invention is to completely encapsulate and insulate the light source so that the sensitive tissue being cauterized is not exposed to any heat generated by the light source.

A yet further object of the present invention is to provide a lighting device that is re-usable and inexpensive.

The above objects and other features and advantages of the present invention, as well as the structure and operation of various embodiments of the present invention, are described in detail below with reference to the accompanying drawings.

### DESCRIPTION OF THE DRAWINGS

The accompanying drawings which are incorporated by reference herein and form part of the specification, illustrate various embodiments of the present invention and, together with the description, further serve to explain the principles of the invention and to enable a person skilled in the pertinent art to make and use the invention. In the drawings, like reference numbers indicate identical or functional similar elements. A more complete appreciation of the invention and many of the attendant advantages thereof will be readily obtained as the same becomes better understood by reference to the following detailed description when considered in connection with the accompanying drawings, wherein:

FIG. 1 is a perspective view of the self-powered lighting system of the present invention in its assembled state as it would be installed on to an electrosurgical pencil.

FIG. 2 is an exploded view of the self-powered lighting system of the present invention.

FIGS. 3a and 3b are perspective views of the lens cover of the present invention.

FIGS. 4a and 4b are perspective views of the main body of the present invention.

FIGS. 5a and 5b are perspective views of the circuit board of the present invention.

FIG. 6 is a perspective view of the metal strip of the present invention.

FIG. 7 is a cross-sectional view of the self-powered lighting system of the present invention in its assembled state as it would be installed on to an electrosurgical pencil.

FIG. 8 is a perspective view of the present invention assembled on to an electrosurgical pencil.

### DETAILED DESCRIPTION OF THE PREFERRED EMBODIMENTS

Reference will now be made to the drawings in which various elements of the present invention will be given numerical designations and in which the invention will be discussed so as to enable one skilled in the art and make use the invention.

The present invention comprises a self-powered lighting system 100 for use with an electrosurgical pencil 200 and having a main body 10, a lens cover 20, a battery 30, a light source 40, a circuit board 50, an attachment ring 60, and an attachment clip 65. It is well known that to accurately perform a surgical procedure with an electrosurgical pencil 200, a medical professional needs to be able to illuminate and view the local tissue area to be cauterized. Thus, it is imperative that effective lighting be provided to the affected regions during the surgical procedure. The self-powered lighting system 100 of the present invention meets the strict waterproofing and sanitizing requirements of a surgery room and allows the affected regions to be properly illuminated.

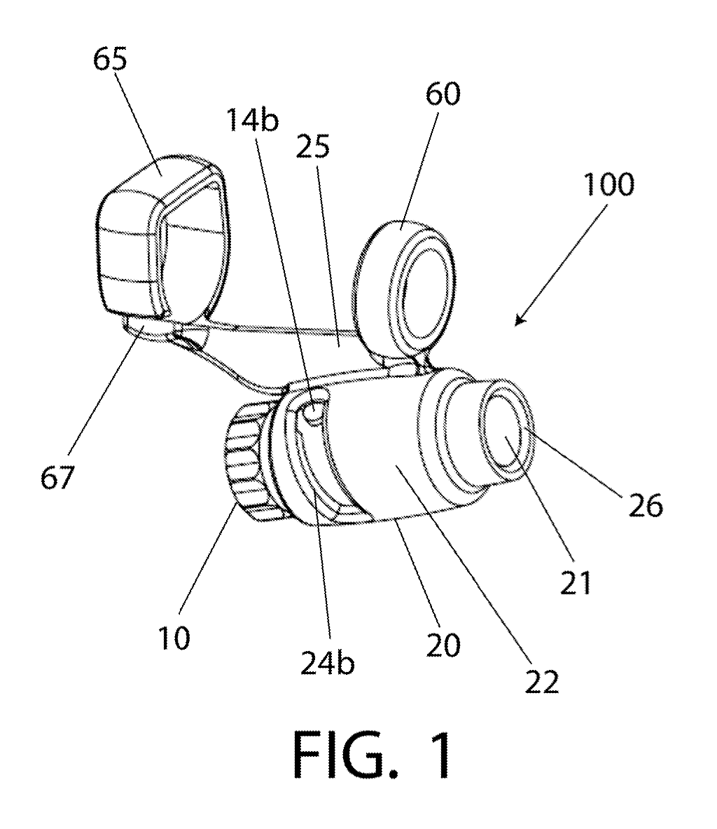

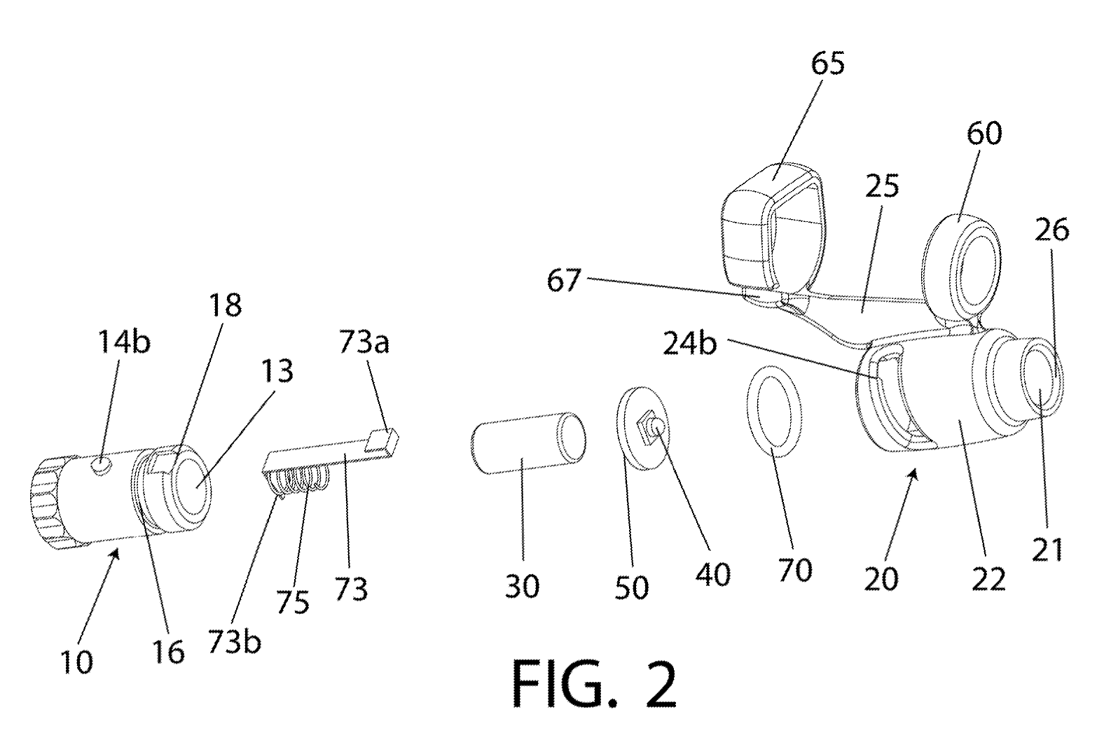

FIG. 1 shows a perspective view of the self-powered lighting system 100 of the present invention in its assembled state as it would be used with an electrosurgical pencil 200. FIG. 2 shows an exploded view of the self-powered lighting system 100 of the present invention to depict in greater detail the various components that comprise the self-powered lighting system 100. The main component of the present invention is the lens cover 20 that rotatably attaches to the main body 10 and that has an attachment mechanism 25 that comprises of the attachment ring 60 and the attachment clip 65 that facilitate the mounting of the self-powered lighting system 100 to the electrosurgical pencil 200.

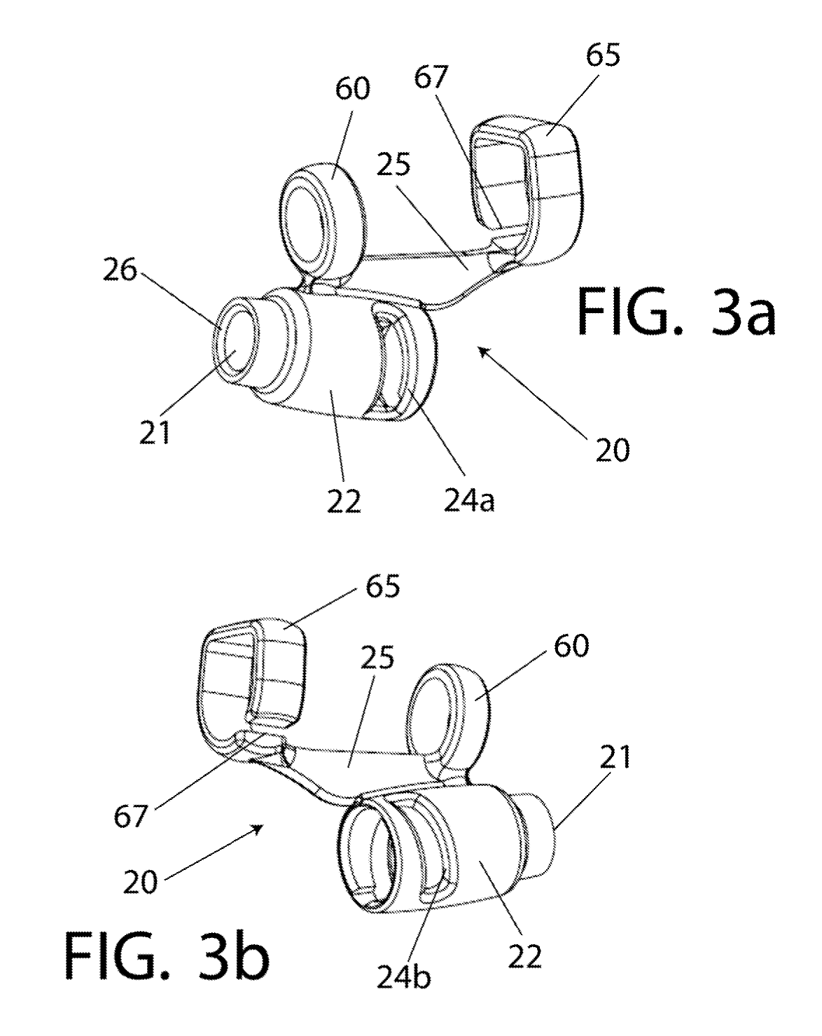

As shown in FIGS. 3a and 3b, the lens cover 20 has a lens 21 from which an annular sidewall 22 extends to define an open cavity 23. The entire lens cover 20 is manufactured out of a clear plastic. The annular sidewall 22 has two slots 24a and 24b that are on opposite sides to each other and that are skewed at equal but opposite angles to each other. Thus, the first slot 24a runs vertically and is skewed so that the top end is further forward towards the lens 21 than the bottom end, as shown in FIG. 3a. On the other hand, the second slot 24b also runs vertically but is skewed so that the top end is further back away from the lens 21 than the bottom end, as shown in FIG. 3b.

Furthermore, an attachment mechanism 25 extends from the top side of the annular sidewall 22 and branches out into the attachment ring 60 and the attachment clip 65. The attachment ring 60 is positioned in front of the attachment clip 65, thus, closer to the lens 21. Both the attachment ring 60 and the attachment clip 65 are used together to mount the self-powered lighting system 100 of the present invention to the electrosurgical pencil 200. Finally, a small annular front ring 26 extends for a very short distance from the lens 21 in the opposite direction as the annular sidewall 22.

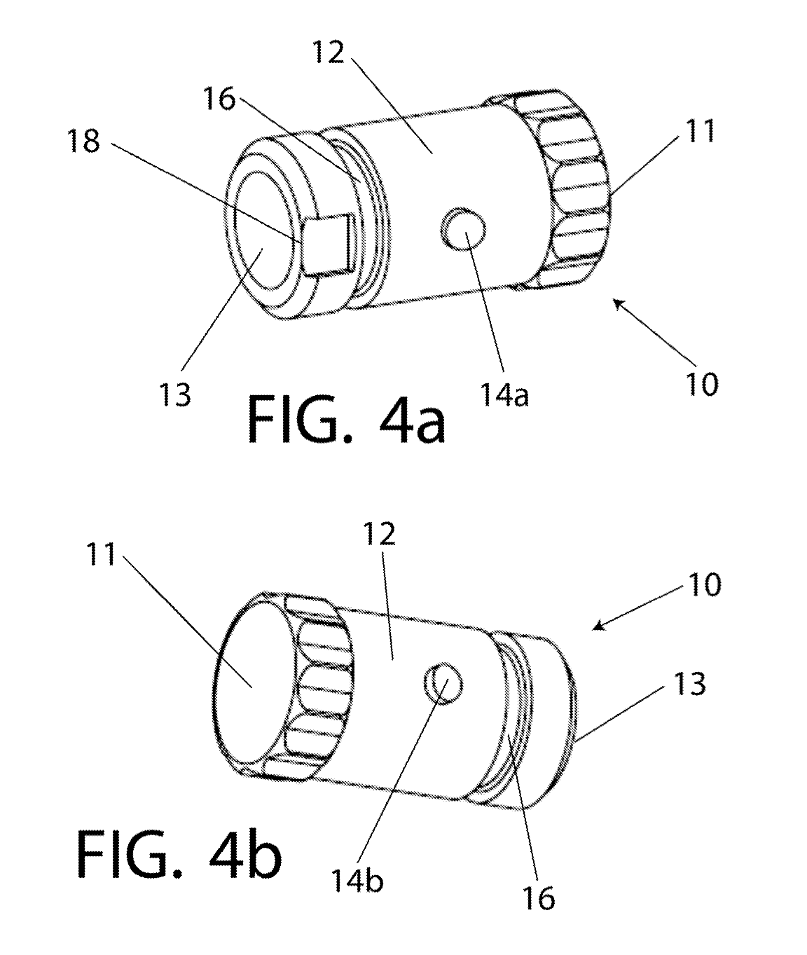

As shown in FIGS. 4a and 4b, the main body 10 has a back plate 11 from which an annular sidewall 12 extends to define an open cavity 13. Two circular pegs 14a and 14b extend out from the annular sidewall 12 and are of sufficient size to fit within the slots 24a and 24b of the lens cover 20. Finally, a small annular channel 16 is created in the annular sidewall 12 into which a small rubber grommet or O-ring 70 is placed.

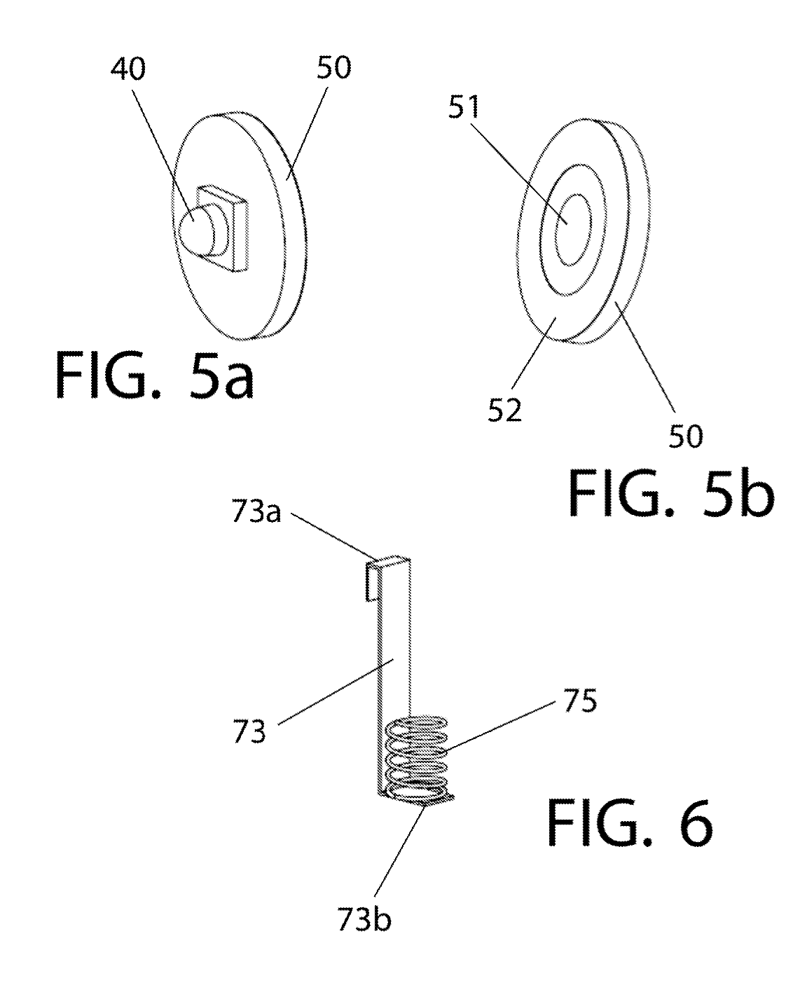

The battery 30 is a standard alkaline battery that is small in size. The light source 40 is at least one LED diode that is high-powered and emanates white light. However, the inventor prefers to use two LED diodes as the light source 40 so as to increase the brightness and intensity of the light. The circuit board 50 has the light source 40 attached to one side. The opposite side of the circuit board 50 has a first metal contact 51 centrally located and electrically connected to the first polar side of the light source 40. This side of the circuit board 50 also has a separate second metal contact 52 around the periphery that is electrically connected to the second polar side of the light source 40, as shown in FIGS. 5a and 5b.

Finally, the preferred embodiment of the present invention has a metal strip 73 that is narrow and having a top end 73a and a bottom end 73b, as shown in FIG. 6. The metal strip 73 is a single piece of thin metal cut into a long rectangular shape having a narrow width. The bottom end 73b is a piece of the thin metal bent at a single 90 degree angle relative to the rest of the metal strip 73. The top end 73a is a piece of the thin metal bent at 90 degrees twice to create a U-shape configuration, as shown in FIG. 6.

Hereinafter, an explanation on the methods of assembling the product of the present invention, the installation thereof to an electrosurgical pencil 200, and the operating states thereof will be given.

Assembly of the product of the present invention begins by placing the small rubber grommet or O-ring 70 into the small annular channel 16 that is in the annular sidewall 12 of the main body 10. Then the bottom end 73b of the metal strip 73 is pushed into the cavity 13 of the main body 10 until the U-shape configuration of the top end 73a of the metal strip 73 wraps around the rim 18 of the annular sidewall 12 of the main body 10. The preferred embodiment is sized so that the top end 73a of the metal strip 73 wraps around the rim 18 when the bottom end 73b abuts against the back plate 11.

Thereafter, a metal spring 75 is dropped into the cavity 13 of the main body 10 until it rests against the bottom end 73a of the metal strip 73. Thus, the metal strip 73 is electrically connected to the metal spring 75. Then the battery 30 is placed into the cavity 13 until it rests against the metal spring 75. Since the components are made of metal, the polar side of the battery 30 that is in physical contact with the metal spring 75 is electrically connected to the thin metal strip 73.

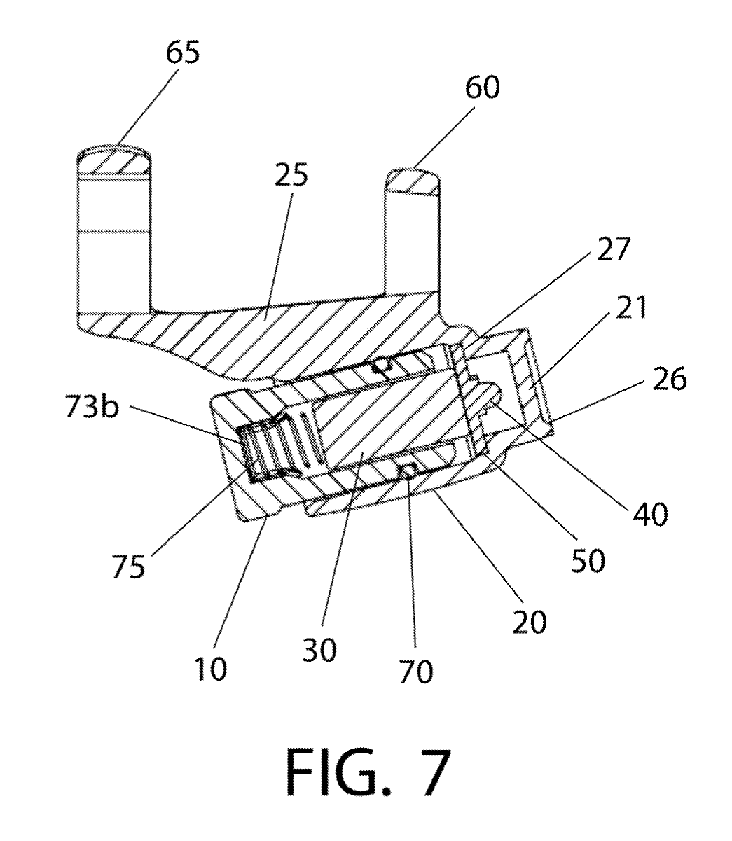

The circuit board 50 is then pushed into the cavity 23 of the lens cover 20 with the light source 40 facing toward the lens 21. The circuit board 50 is pushed into the cavity 23 until it rests against a support ledge 27 that prevents the light source 40 from being pressed against the lens 21. With the circuit board 50 in place inside the cavity 23, the end of the main body 10 opposite to the back plate 11 is pushed into the open cavity 23 of the lens cover 20 until the two circular pegs 14a and 14b of the main body 10 are within the two corresponding slots 24a and 24b of the lens cover 20. When the pegs 14a and 14b are within the slots 24a and 24b, the assembly of the self-powered lighting system 100 of the present invention is completed, as shown in FIG. 7.

Once the assembly of the present invention is completed, operating the self-powered lighting system 100 is not complicated.

When the assembly of the self-powered lighting system 100 is completed, the battery 30 is held tightly in between the metal spring 75 and the circuit board 50. The metal spring 75 is pressed against the bottom end 73b of the metal strip 73 that abuts the back plate 11 of the main body 10. When the main body 10 is properly assembled in the lens cover 20, the metal spring 75 is compressed such that the tension of the metal spring 75 pushes the battery 30 against the first metal contact 51 of the circuit board 50. Finally, the circuit board 50 is pressed against the support ledge 27 of the lens cover 20. As such, the first polar side of the battery 30 is electrically connected to the first metal contact 51 in the circuit board 50 due to their physical connection facilitated by the tension of the metal spring 75. Similarly, a second polar side of the battery 30 is electrically connected to the metal strip 73 via the metal spring 75.

When the main body 10 is properly assembled in the lens cover 20, the pegs 14a and 14b that are within the slots 24a and 24b allow the main body 10 to be rotated within the lens cover 20 by a small amount. Thus, the main body 10 can be rotated clockwise or counter-clockwise within the lens cover 20. The angularity of the slots 24a and 24b facilitate the forward and backward displacement of the main body 10 within the lens cover 20 as it is rotated. As such, when the main body 10 is rotated clockwise, it is displaced forward or deeper into the lens cover 20. Similarly, when the main body 10 is rotated counter-clockwise, it is displaced backward or out of the lens cover 20. The amount of rotation and amount of displacement of the main body 10 relative to the lens cover 20 is dictated by the length and angularity of the slots 24a and 24b.

The length and angularity of the slots 24a and 24b of the preferred embodiment of the present invention are designed so that the main body 10 is displaced a sufficient distance to electrically connect and electrically disconnect the metal strip 73 and the second metal contact 52 in the circuit board 50. Accordingly, when the main body 10 of the preferred embodiment of the present invention is rotated clockwise, the main body 10 is displaced enough to bring the top end 73a of the metal strip 73 in physical contact with the second metal contact 52 in the circuit board 50. Similarly, when the main body 10 is rotated counter-clockwise, it is displaced enough to physically separate the top end 73a of the metal strip 73 from the second metal contact 52 in the circuit board 50.

Therefore, since the first polar side of the battery 30 is already electrically connected to the first metal contact 51 in the circuit board 50 due to their physical connection facilitated by the tension of the metal spring 75, the clockwise rotation of the main body 10 facilitates the electrical connection of the second polar side of the battery 30 with the second metal contact 52 in the circuit board 50. This closes the electrical circuit and the light source 40 is energized to illuminate or emanate light. On the other hand, the counter-clockwise rotation of the main body 10 electrically disconnects the second polar side of the battery 30 from the second metal contact 52 in the circuit board 50. This opens the electrical circuit and the light source 40 is turned off.

In other words, the simple rotation of the main body 10 relative to the lens cover 20 turns on or off the light being emanated by the self-powered lighting system 100 of the present invention. There are no complicated buttons or switches. It is the simple rotation of the main body 10 that electrically controls the present invention.

The preferred embodiment of the present invention is configured to be controlled by the rotation of the main body 10 rather than by the rotation of the lens cover 20 because rotation of the lens cover 20 would require the user to touch and grip it, thus increasing the likelihood that the lens 21 can be clouded or stained with fingerprints. The main body 10 is manufactured of an opaque plastic material thus its clarity is not critical to the quality of light emanating from the self-powered lighting system 100.

Once the self-powered lighting system 100 of the present invention is assembled and operational, it is mounted on to an electrosurgical pencil 200 with the attachment ring 60 and the attachment clip 65.

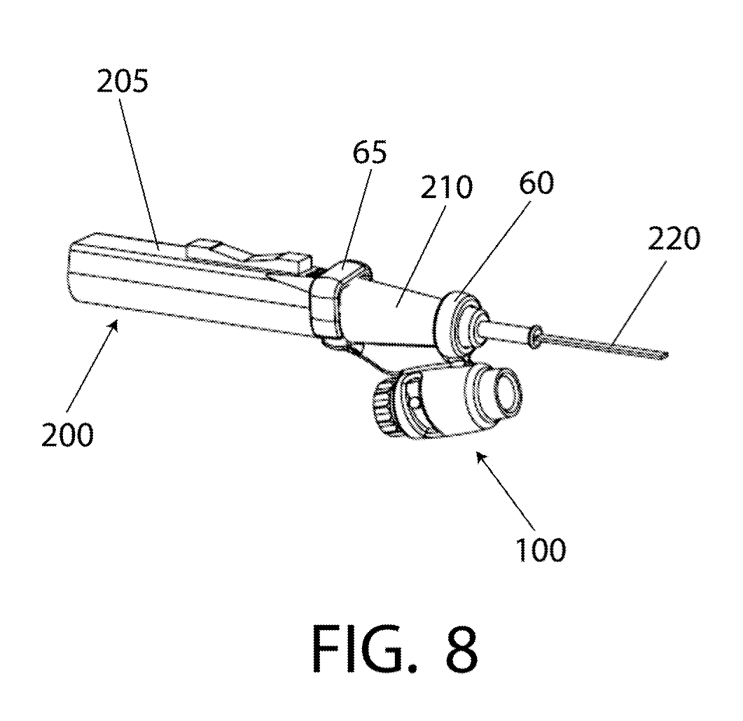

An electrosurgical pencil 200 comprises a long narrow handle 205 having a conically-shaped tip 210 to which an electrode 220 is attached, as shown in FIG. 8. The electrode 220 is that piece that cuts or cauterizes tissue. The handle 205 and tip 210 are insulated to allow the user to grip or hold the instrument therefrom. Mounting the self-powered lighting system 100 to the electrosurgical pencil 200 is quick and simple. The electrode 220 is passed through the attachment clip 65 and then through the attachment ring 60 until the self-powered lighting system 100 is snuggly mounted and held in place around the electrosurgical pencil 200.

The attachment ring 60 is sized so that it is larger in diameter than the small end of the conically-shaped tip 210 but smaller in diameter than the large end of the conically-shaped tip 210. Thus, when properly mounted, the attachment ring 60 is wrapped around the middle section of the conically-shaped tip 210. When the attachment ring 60 is in place, the attachment clip 65 is wrapped around the front end of the handle 205.

The attachment ring 60 is circular in shape so as to match the circularity of the conically-shaped tip 210. On the other hand, the attachment clip 65 takes the shape of a rounded square to match the shape of the handle 205. The electrosurgical pencil 200 has a handle 205 with a rounded square shape to facilitate the user to maintain a steady grip or hold of the device during the surgical procedure. A circular handle would allow the inadvertent rotation of the electrosurgical pencil 200 during a surgical procedure. However, the flat surfaces of the rounded square handle 205 allow the user to grip the device with a lessened opportunity to inadvertently rotate it during the procedure. This is a very important feature of electrosurgical pencils. Thus, the attachment clip 65 takes the shape of the handle 205 to allow a matching fit to the handle 205 and to prevent the self-powered lighting system 100 from inadvertently rotating around the handle 205 during a surgical procedure. Furthermore, due to the rounded square shape, the self-powered lighting system 100 can be positioned on the electrosurgical pencil 200 in any one of four positions: underneath, above, or either side of the electrode 220. The position of the self-powered lighting system 100 can be switched without having to completely remove it from the electrosurgical pencil 200. Instead, the self-powered lighting system 100 is pulled forward just until the attachment clip 65 is not around the handle 205 but around the conically-shaped tip 210. Then the self-powered lighting system 100 is rotated in 90 degree increments until it is in the desired position. Finally, the self-powered lighting system 100 is pushed back until the attachment clip 65 is around the handle 205.

As discussed above, the attachment ring 60 is shaped as a closed circle. However, the attachment clip 65 is not closed all around. Instead it has a gap or an opening 67 that promotes structural flexibility. In fact, the preferred embodiment of the present invention has an attachment clip 65 that is slightly undersized relative to the size of the handle 205. Thus, the attachment clip 65 is flexed outward when it is wrapped around the handle 205. The outward flexion allows pressure to be exerted by the attachment clip 65 against the handle 205. The pressure is sufficient to allow the attachment clip 65 to grip the handle 205 without slippage. As such, the attachment clip 65 is designed to grip and prevent the self-powered lighting system 100 from slipping off the electrosurgical pencil 200 inadvertently during a surgical procedure.

The manner by which the self-powered lighting system 100 of the present invention is mounted to the electrosurgical pencil 200 is unique and different than the prior art and the '180 patent, the closest prior art to the present invention found by the inventor. The '180 patent teaches the use of loop-and-hook VELCRO™, flexible strip, flanges, and suction cups as ways to mount the illumination device to any surgical instrument. Thus, the '180 patent broadly teaches an illumination device that can be mounted in a variety of ways on any surgical instrument. On the other hand, the self-powered lighting system 100 of the present invention can be mounted specifically to an electrosurgical pencil and specifically using a unique attachment clip 65 and attachment ring 60 to prevent inadvertent movement during a surgical procedure.

Furthermore, the '180 patent teaches an illumination device with the light source or the LED diode outside the housing and exposed to the elements. This allows the heat emanating from the light source to pose a risk of burning or injuring the sensitive tissue that is being cut or cauterized in a surgical procedure. On the other hand, the self-powered lighting system 100 of the present invention has a light source 40 that is completely encapsulated and insulated within the lens cover 20.

In addition, the self-powered lighting system 100 of the present invention only has two components exposed to the outside elements, the lens cover 20 and the main body 10. All other parts and components are encapsulated and tightly sealed inside the present invention. This configuration allows the present invention to be properly sanitized to meet the strict standards and requirements of a surgery room setting. On the other hand, the '180 patent teaches an illumination device that has several components exposed to the outside elements. The '180 patent device has the entire housing and the light source completely exposed outside. The light source, especially, is difficult to sanitize since it is an electronic component. Additionally, the '180 patent device has a switching mechanism to turn on the light that can be a non-conductive element that is pulled out of the housing, a membrane button, or a switch. In essence, the '180 patent is designed to expose the switching mechanism to the outside which becomes problematic since these components are electronic in nature and difficult to sanitize. Finally, the '180 patent device has an attachment mechanism that can be VELCRO™, suction cups, or other components that are exposed outside to the elements and nearly impossible to sanitize. In all, the self-powered lighting system 100 of the present invention is uniquely designed to only have two plastic non-electronic components exposed to the outside elements. Thus, the present invention is superior to the '180 patent in that it can be sanitized more effectively to meet the strict requirements of a surgery room setting.

Thirdly, the self-powered lighting system 100 of the present invention is designed to be water-resistant so as to protect against blood or other fluids to which it is exposed in a surgery room setting. Such fluids making contact with the electronics of the device can damage the device and compromise the surgical procedure. The rubber grommet or O-ring 70 between the lens cover 20 and the main body 10 creates a barrier to prevent fluids from penetrating into the light source 40, the battery 30, the circuit board 50, or the metal strip 73. On the other hand, the illuminating device taught by the '180 patent has a light source completely exposed and accessible by the fluids. In addition, the '180 patent has a non-conductive element that is pulled out of the housing to turn on the light. Thus, when the non-conductive element is removed, an unprotected path for fluid to leak into the inside of the housing is automatically created. As such, unlike the present invention, the '180 patent is not water-resistant at all whatsoever.

Finally, the device taught by the '180 patent has a light source outside of the housing and without any lensing. On the other hand, the present invention has a lens cover 20 that has a lens 21 designed to complement the distribution of light from the light source 40. The lens cover 20 also has a small annular front ring 26 extending a short distance from the lens 21 and designed to aid in directing the light to illuminate the affected area of the surgical procedure.

Therefore, the self-powered lighting system 100 of the present invention is superior in design and functionality than any existing prior art, especially the '180 patent. It is understood that the described embodiments of the present invention are illustrative only, and that the modifications thereof may occur to those skilled in the art. Accordingly, this invention is not to be regarded as limited to the embodiments disclosed, but to be limited only as defined by the appended claims herein.

What is claimed is:

1. A self-powered lighting system that is detachably mountable to an electrosurgical pencil comprising:
a lens cover having at least one slot;
a light source within said lens cover;
a main body having at least one peg;
said main body that is rotatably attached to said lens cover with said peg extending through said slot such that said main body can be rotated relative to said lens cover a predetermined distance defined by said slot and said peg;
a battery within said main body;
a circuit board that electrically connects said battery to said light source when said main body is rotated in a first direction relative to said lens cover a predetermined distance;
said circuit board that electrically disconnects said battery from said light source when said main body is rotated in a second direction relative to said lens cover a predetermined distance;
an attachment mechanism extending from said lens cover; and
wherein said attachment mechanism enables said self-powered lighting system to be mounted to said electrosurgical pencil.

2. The self-powered lighting system of claim 1 further comprising a rubber grommet placed between said main body and said lens cover to keep liquid substances away from said circuit board, said light source, and said battery.

3. The self-powered lighting system of claim 1 wherein said lens cover and said main body are made of plastic material that can be sanitized.

4. The self-powered lighting system of claim 1 further comprising a front ring that extends from said lens cover and that aids in directing light to a predetermined area.

5. The self-powered lighting system of claim 1 wherein said attachment mechanism comprises an attachment ring through which an electrode on said electrosurgical pencil is inserted and an attachment clip having a gap that promotes structural flexibility to allow said attachment clip to be wrapped around a handle on said electrosurgical pencil.

6. The self-powered lighting system of claim 5 wherein said attachment clip has a shape that is substantially similar to the shape of said handle on said electrosurgical pencil.

7. A self-powered lighting system that is detachably mountable to an electrosurgical pencil comprising:
a lens cover;
a light source within said lens cover;
a main body that is rotatably attached to said lens cover;
a battery within said main body;
a circuit board that electrically connects said battery to said light source when said main body is rotated in a first direction relative to said lens cover;
said circuit board that electrically disconnects said battery from said light source when said main body is rotated in a second direction relative to said lens cover;
an attachment mechanism that extends from said lens cover; and
wherein said attachment mechanism enables said self-powered lighting system to be mounted to said electrosurgical pencil.

8. The self-powered lighting system of claim 7 further comprising a rubber grommet placed between said main body and said lens cover to keep liquid substances away from said circuit board, said light source, and said battery.

9. The self-powered lighting system of claim 7 wherein said lens cover and said main body are made of plastic material that can be sanitized.

10. The self-powered lighting system of claim 7 further comprising a front ring that extends from said lens cover and that aids in directing light to a predetermined area.

11. The self-powered lighting system of claim 7 wherein said attachment mechanism comprises an attachment ring through which an electrode on said electrosurgical pencil is inserted and an attachment clip having a gap that promotes structural flexibility to allow said attachment clip to be wrapped around a handle on said electrosurgical pencil.

12. The self-powered lighting system of claim 11 wherein said attachment clip has a shape that is substantially similar to the shape of said handle on said electrosurgical pencil.

13. A self-powered lighting system that is detachably mountable to an electrosurgical pencil comprising:
a lens cover containing a light source;
a main body containing a battery and rotatably attached to said lens cover;
a switchable circuit through which said light source is powered when said main body is rotated a predetermined distance relative to said lens cover; and
an attachment mechanism that extends from said lens cover that enables said self-powered lighting system to be mounted to said electrosurgical pencil.

14. The self-powered lighting system of claim 13 further comprising a rubber grommet placed between said main body and said lens cover to keep liquid substances away from said circuit board, said light source, and said battery.

15. The self-powered lighting system of claim 13 wherein said lens cover and said main body are made of plastic material that can be sanitized.

16. The self-powered lighting system of claim 13 further comprising a front ring that extends from said lens cover and that aids in directing light to a predetermined area.

17. The self-powered lighting system of claim 13 wherein said attachment mechanism comprises an attachment ring through which an electrode on said electrosurgical pencil is inserted and an attachment clip having a gap that promotes structural flexibility to allow said attachment clip to be wrapped around a handle on said electrosurgical pencil.

18. The self-powered lighting system of claim 17 wherein said attachment clip has a shape that is substantially similar to the shape of said handle on said electrosurgical pencil.
